(19) United States
(12) Patent Application Publication
(10) Pub. No.: US 2026/0096743 A1
(43) Pub. Date: Apr. 9, 2026

(54) DETERMINING RESPIRATORY PHASE INFORMATION

(71) Applicant: INSPIRE MEDICAL SYSTEMS, INC., Golden Valley, MN (US)

(72) Inventors: Maxwell P. Lundeen, Minneapolis, MN (US); Gerardo Rodriguez Orellana, Minneapolis, MN (US)

(73) Assignee: INSPIRE MEDICAL SYSTEMS, INC., Golden Valley, MN (US)

(21) Appl. No.: 19/351,123

(22) Filed: Oct. 6, 2025

Related U.S. Application Data
(60) Provisional application No. 63/703,889, filed on Oct. 4, 2024.

(51) Int. Cl. A61B 5/08; A61B 5/00; A61B 5/087

(52) U.S. Cl. CPC A61B 5/0816; A61B 5/0022; A61B 5/087; A61B 5/4815; A61B 5/7267; A61B 5/7282; A61B 5/7435

(57) ABSTRACT

A method includes receiving, at a first service provider, a sensed respiratory signal of a first respiratory sensing session. At or via the first service provider, the method includes determining respiratory phase information based on the received sensed respiratory signal.

### BACKGROUND

A significant portion of the population suffers from various forms of sleep-related issues, some of which may involve sleep disordered breathing (SDB) and/or other conditions. At least because sleep disordered breathing (SDB) may primarily be a respiratory-related phenomenon, effective respiratory sensing is desirable.

### BRIEF DESCRIPTION OF THE DRAWINGS

FIG. 1 is a block diagram representing an example method of determining respiratory phase information.

FIG. 2 is a block diagram representing an example method of automatically determining respiratory phase information based on a first respiratory sensing session.

FIG. 3 is a block diagram representing an example method of constructing a data model.

FIG. 4 is a flow diagram representing an example method of constructing a data model.

FIG. 5A is a flow diagram representing an example method of determining respiratory phases using a constructed a data model.

FIG. 5B is block diagram representing example outputs including respiratory phase transitions.

FIG. 6 is a block diagram representing an example data model portion.

FIG. 7 is a diagram including a graph representing an example unlabeled respiratory signal.

FIG. 8 is a diagram including a graph representing example unprocessed human labeling for a respiratory signal.

FIG. 9 is a diagram including a graph representing human labeling juxtaposed with the example respiratory signal of FIG. 7.

FIG. 10 is a flow diagram representing an example method of training (e.g. constructing) a data model for determining at least respiratory phase information.

FIG. 11 is a diagram including a graph representing example smoothed labeling for a respiratory signal.

FIG. 12 is a flow diagram representing an example method of training using a constructed a data model for determining at least respiratory phase information.

FIG. 13 is a diagram including a graph representing an example method of selecting labels for transitions between phases of a sensed respiratory signal.

FIG. 14A is a diagram including a graph representing human labeling and automated labeling of respiratory phase transitions applied to the example respiratory signal of FIG. 7.

FIG. 14B is a block diagram representing example methods which use the outputs determined from applying an example constructed data model to current inputs.

FIG. 15A is a diagram schematically representing an example patient care environment.

FIGS. 15B-15F are diagrams schematically representing methods and/or aspects of methods of determining respiratory phase information based on a respiratory signal.

FIG. 16A is a block diagram schematically representing an example control portion.

FIG. 16B is a diagram schematically illustrating at least some example arrangements of a control portion.

FIG. 16C is a block diagram schematically representing a user interface.

FIG. 17 is a block diagram which schematically represents some example implementations by which an IMD may communicate wirelessly with external devices outside the patient.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense. It is to be understood that features of the various examples described herein may be combined, in part or whole, with each other, unless specifically noted otherwise.

While the use of sleep studies such as polysomnography (PSG) have been present for a considerable period of time, the process to accurately label breaths in respiratory signals has remained time-consuming and laborious because this labeling is generally still performed manually by specially trained technicians. These labels are used for distinguishing between the inspiration and expiration phases of breathing, which is significant in clinical settings where precise respiratory monitoring may be essential for certain diagnoses and treatment decisions. While some prior attempts have been made to automatically label breaths (in a respiratory signal obtained in a sleep study), these prior attempts have failed to address the problem of signal drift and noise, have not been scalable, and/or such prior attempts were not trained on patients having obstructive sleep apnea. In some instances, such labeling may sometimes be referred to as identifying respiratory phase information, including identifying an inspiration phase, an expiration phase, and/or an expiration pause phase. Such identification of the respective phases may comprise identifying a transition between the inspiration phase and the expiration phase, a transition between the expiration phase and the expiration pause phase, and/or a transition between the expiration pause phase and the inspiration phase.

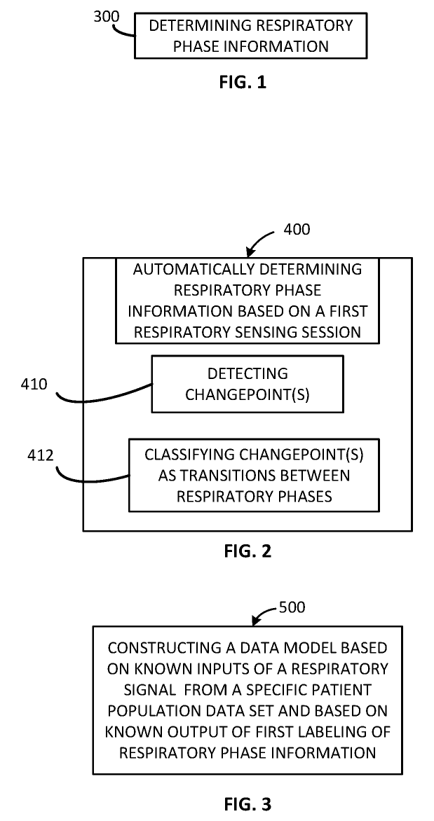

Accordingly, as shown at 300 in FIG. 1 at least some example methods 300 of the present disclosure may comprise determining respiratory phase information in novel ways. In some examples, such methods may be implemented via a device including (but not limited to) a control portion or service provider configured to (e.g. programmed to) determine the respiratory phase information according to at least some of the features of the various examples throughout the present disclosure.

As shown in FIG. 2, one example implementation of method 300 (FIG. 1) includes an example method 400 comprising determining (e.g. automatically) respiratory phase information based on a first respiratory sensing session. In some such examples, the first respiratory sensing session may comprise a formal sleep study such as (but not limited to) a polysomnography session in which respiratory sensing is performed such as via airflow sensing (e.g. via the nostrils). In some examples, the first respiratory sensing session (e.g. PSG session) also may comprise sensing respiratory effort, which may be performed via sensing at or via the torso (e.g. chest, abdomen) and/or other body portions. In some examples, the sensed respiratory effort may supplement and/or be used instead of the airflow sensing to determine respiratory phase information. However, in general terms the airflow sensing may generally be considered a more accurate, robust method of determining respiratory phase information.

In some examples, the method 400 may comprise detecting changepoints (at 410) and may comprise classifying the detected changepoints (at 412). For instance, detecting a change point (410) may comprise determining a change in direction (and magnitude) of the sensed respiratory signal, with such determined directional change being indicative of a change from one respiratory phase to another respiratory phase. In some such examples, the determined magnitude of the change may be compared to a selectable threshold to determine if the change is of a sufficient magnitude to deem the change in direction of the sensed respiratory signal to indicate a bona fide change from one respiratory phase to another different respiratory phase.

In some examples, once a changepoint has been detected (410), at 412 method 400 may comprise classifying the changepoint as a transition in the respiratory signal. In some such examples, the transition may comprise a first transition from inspiration to expiration, a second transition from expiration to expiration pause, and/or a third transition from expiration pause to inspiration.

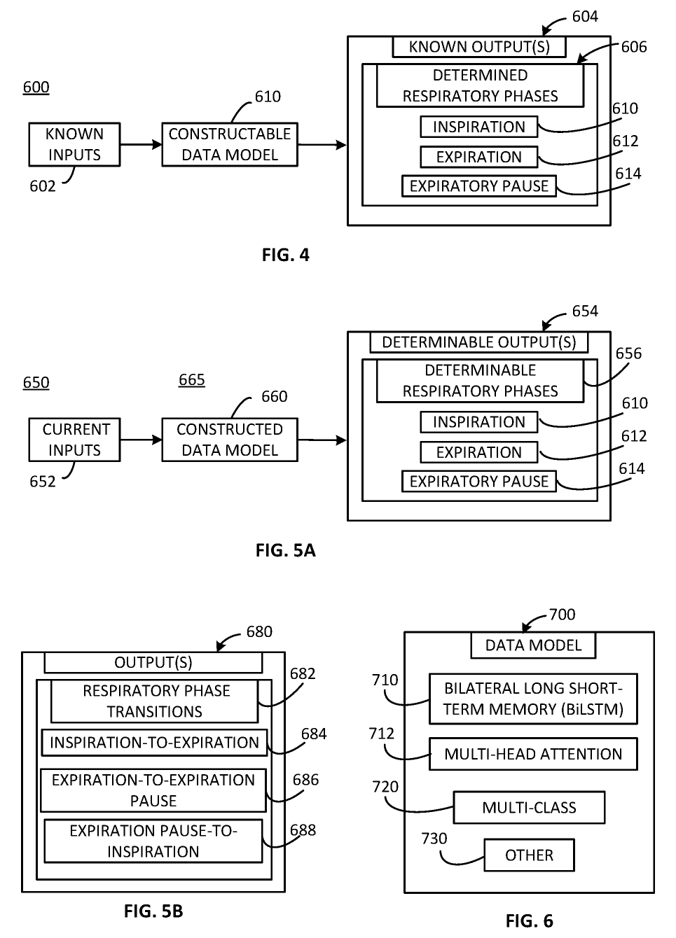

As shown in FIG. 3, one example method 500 comprises constructing a data model based on known inputs (e.g. 602 in FIG. 4) of a respiratory signal from a specific patient population data set and based on known outputs (e.g. 604 in FIG. 4) of a first labeling of respiratory phase information. In some such examples, the first labeling may comprise labeling one or more of the above-identified transitions between respiratory phases. In some of these examples, the first labeling may comprise human labeling of the respiratory phase information for which an example is illustrated later in FIG. 8.

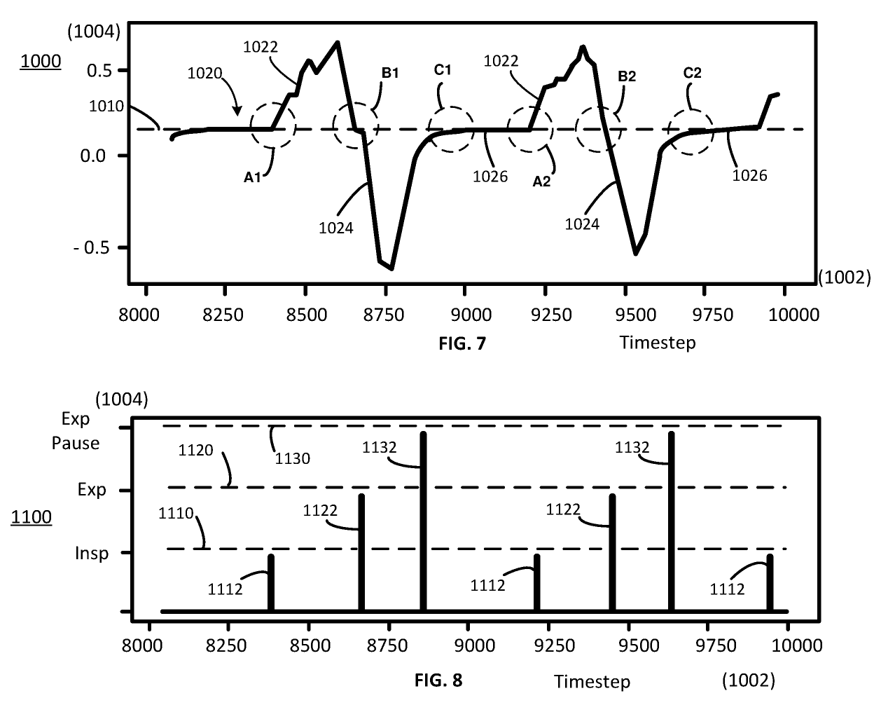

In some examples, the known inputs in method 500 (e.g. see also 602 in FIG. 4) may comprise a highly accurate, robust respiratory signal (e.g. FIG. 7) such as (but not limited to) obtained via the above-described first respiratory sensing session. Meanwhile, in some examples the specific patient population data set may comprise data from a group of patients for which various parameters relating to sleep, respiration, cardiac, etc. have been sensed, identified, and classified, and which therefore may be used as a robust, detailed example suitable to recognize characteristics of a transitions between respiratory phases in a sensed respiratory signal (e.g. airflow respiratory signal).

With this in mind, in some examples the known outputs of method 500 in FIG. 3 (see also 604 in FIG. 4) may comprise human labeling (e.g. FIG. 8) which was applied in the specific patient population data set of the different transitions (e.g. inspiration to expiration, expiration to expiratory pause, and/or expiratory pause to inspiration). In some examples throughout the present disclosure, the terms inspiration and inspiratory may be used interchangeably and/or the terms expiration and expiratory may be used interchangeably such as (but not limited to) when referring to an inspiration phase (e.g. inspiratory phase), an expiration phase (e.g. expiratory phase), or an expiration pause phase (e.g. expiratory pause phase).

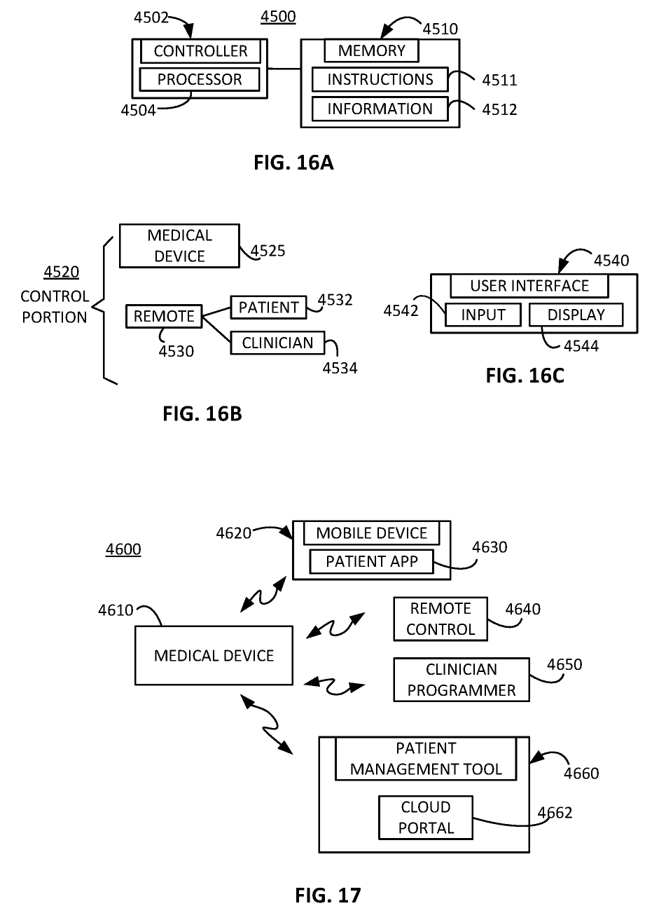

In some examples, various information relating to the example methods may be displayed via a user interface (e.g. 4540 in FIG. 16C), such as (but not limited to) displaying known outputs and/or known inputs of a constructable data model during training, displaying outputs and/or inputs of a constructed data model before, during, and/or after use of the data model. As further described later, the user interface also may be used to display other information such as (but not limited to) adjustable settings for sensing (e.g. automatic respiratory sensing) and/or therapy stimulation, upper airway collapse determination, etc.

FIG. 4 illustrates one example implementation of at least the method 500 of FIG. 3 of constructing a data model in which known inputs 602 are supplied to a constructable data model 610 in relation to known outputs 604 such as (but not limited to) already determined respiratory phases which may comprise inspiration 610, expiration 612, and expiratory pause 614.

FIG. 5A illustrates one example method 650 which exploits the example methods 500 (FIG. 3), 600 (FIG. 4) by supplying current inputs 652 to a constructed data model 660 to produce determinable outputs 654, which may comprise determinable respiratory phases 656 (including 610, 612, 614).

FIG. 5B is block diagram representing example outputs including respiratory phase transitions. As shown 680 in FIG. 5B, in some examples of the various data models throughout the present disclosure, the output(s) by which a data model may be constructed (and/or the outputs determinable via application of a constructed data model) may comprise respiratory phase transitions 682, which may comprise a inspiration-to-expiration transition 684, an expiration-to-expiration pause transition 686, and/or an expiration pause-to-inspiration transition 688.

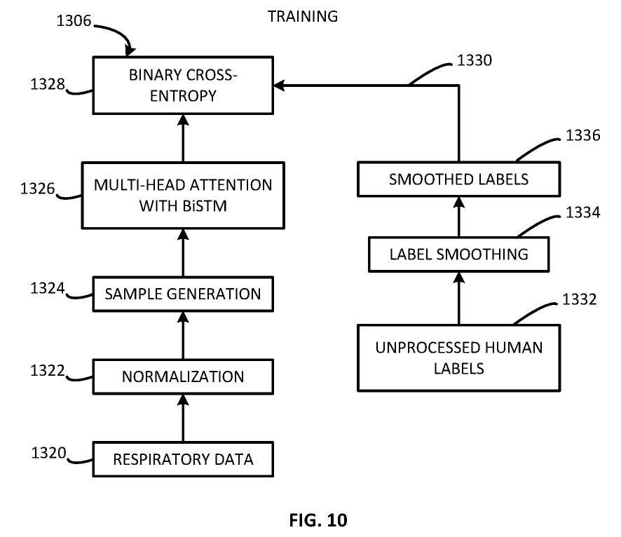

As shown in FIG. 6, an example data model portion 700 may comprise data models by which the example methods of the present disclosure may be implemented. For instance, one example data model may comprise a bilateral long short-term memory (BiLSTM) data model 710 and/or a multi-head attention (MHA) data model 712. In some examples, the BiLSTM data model 710 and multi-head attention (MHA) data model 712 may be implemented together in a complementary manner, which is further illustrated later at 1326 in the example method 1306 depicted in diagram 1300 of FIG. 10.

In one aspect, the BiLSTM data model 710 may predominantly identify changepoints in a time-series or time-based signal, while in another aspect, the multi-head attention data model 712 may, at least partially, implement classification according to multiple classes (e.g. different types) (720) of changepoints. In at least some of the examples of the present disclosure, the multiple classes (e.g. classifications) of changepoints correspond to the different transitions within the sensed respiratory signal including the transition from inspiration to expiration, transition from expiration to expiratory pause, and transition from expiratory pause to inspiration. In some such examples, the multi-head attention data model 712 may enhance the multiple classification at least because the accuracy and robustness of the determination (e.g. of the classification of the detected multiple different types of transitions) is generally not impacted by distance between the data points. Moreover, in some examples, the Multi-head attention data model 712 may enhance the multiple classification at least because this data model may help to determine a context in which a particular signal pattern (e.g. detected changepoint (e.g. respiratory signal transition)) appears, which may enhance the robustness and accuracy of classifying a particular changepoint as one of the distinct respiratory transitions noted above.

In addition to (or instead of) using the multi-head attention (MHA) data model 712 to at least partially perform the classification, some example methods also may comprise using a linear layer (e.g. a linear neural network) to at least partially perform the classification.

In some examples, parameter 730 comprises other data models which may be used to identify changepoints while also providing classifying the various changepoints into several different classes of changepoints. In some such examples, at least some deep learning solutions may be used to detect changepoints (e.g. 410 in FIG. 2) and classifying the changepoints (e.g. 412 in FIG. 2) may comprise a Recurrent Neural Network data model, a BiLSTM data model without a Multi-Head attention component, a Convolutional Neural Network (CNN) data model, a Multi-Head Attention data model without a BiLSTM component, or a combination of at least some of these data models (e.g. BiLSTM and CNN combination).

In one aspect, the example data models are scalable such that the data models can be run on large amounts of data in parallel. Moreover, the example data models can be implemented with whatever subset of patient information is available at any particular timeframe the data model is constructed and used. Accordingly, at least some of these examples of the present disclosure represent an advancement in the field of respiratory monitoring, offering a more reliable, scalable, and efficient tool than other previously attempted techniques.

As shown in FIG. 7, a diagram 1000 includes a graph mapping a respiratory signal 1020 according to an x-axis 1002 representing a time-series (e.g. timestamp) and a y-axis 1004 of signal amplitude by which one can identify an inspiration phase 1022, expiration phase 1024, and expiratory pause 1026 relative to a signal threshold/baseline (dashed line 1010). Dashed circles A1, A2 identify inspiration onsets (which corresponds to expiratory pause offsets), dashed circles B1, B2 identify expiration onsets (which corresponds to inspiration offsets), and dashed circles C1, C2 represent onset of expiratory pause (which corresponds to expiration offsets). A representation of the respiratory signal 1020 is also depicted at 1320 in FIG. 10 as part of example method 1300 of FIG. 10.

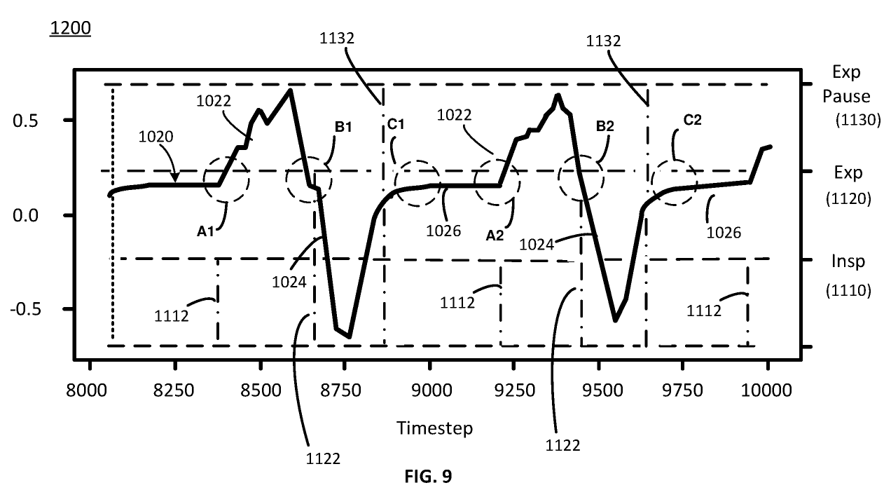

The respiratory signal 1020 may comprise a respiratory signal obtained via a first respiratory sensing session (identified in 400 in FIG. 2), which may be used as a known input (e.g. 602 in FIG. 4) for a data model to be constructed (e.g. trained) with diagram 1100 in FIG. 8 depicting human-determined labels 1112 (for inspiration 1110), 1122 (for expiration 1120), and 1132 (for expiratory pause 1130) with such human labeling serving as a known output (e.g. 604 in FIG. 4) in a constructed data model. This human labeling of respiratory signals (e.g. from a PSG study) is generally considered very time-consuming and laborious. FIG. 9 further depicts the respiratory signal 1020 of FIG. 7, except further showing the human-determined labels of FIG. 8 overlayed onto the respiratory signal 1020 to illustrate the manner in which the human labels identify the changepoints (e.g. transitions) between the different respiratory phases.

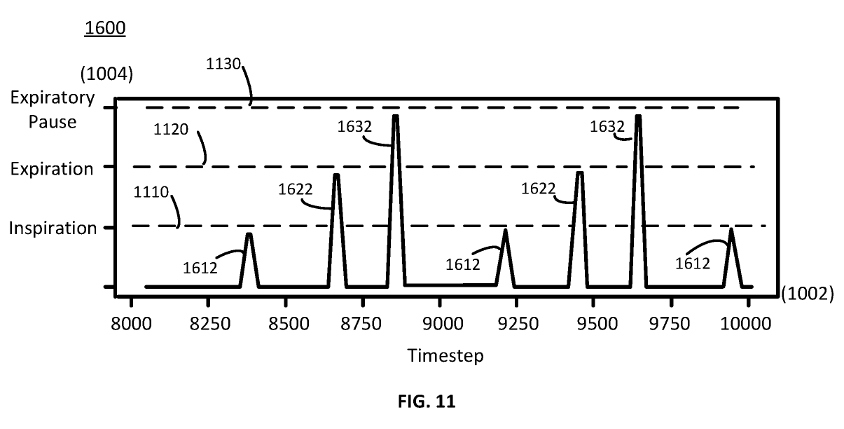

In some examples, continuous smoothing is applied to the human labels (e.g. unprocessed) in diagram 1100 of FIG. 8, which may produce a diagram such as diagram 1600 in FIG. 11 in which each of the various human-determined labels 1112 (inspiration onset), 1122 (expiration onset), and 1132 (expiratory pause onset) in FIG. 9 become transformed into smoothed labels 1612 (inspiration onset), 1622 (expiration onset), and 1632 (expiratory pause onset). In some such examples, the human labels may sometimes be referred to as manual identification (e.g. manual identifying) of the respiratory phase information, and more particularly, manual identification of the inspiration-to-expiration transition, the expiration-to-expiration pause transition, and/or the expiration pause-to-inspiration transition. These smoothed labels (1336) may enhance constructing (e.g. training) a data model per the methods in at least FIGS. 3-5 and as shown in FIG. 10 in which input 1330 comprises smoothed labels 1336, derived via label smoothing (1334) of unprocessed human labels 1332 (shown in FIG. 8).

In one aspect, these smoothed labels may be implemented via preprocessing during training (e.g. constructing the data model) to smooth the discrete and sparse multidimensional labels in high frequency data into a continuous distribution, such that a model prediction close to correct is not punished as much by the loss function (e.g. binary cross entropy 1328) as a model prediction far from correct.

As previously mentioned elsewhere, in some instances such labeling may sometimes be referred to as identifying (e.g. identifications) of the respective respiratory phases, and more particularly, identifying transitions between the respective respiratory phases.

In some examples, the continuous smoothing may be applied to the multidimensional output (e.g. predicting inspiration, expiration, and “expiration pause”).

In some examples, at least some general pre-processing steps for both training (e.g. constructing a data model) and inference (e.g. using a constructed data model) may comprise normalization (e.g. 1322 in FIG. 10) on a per-night basis and splitting into windows.

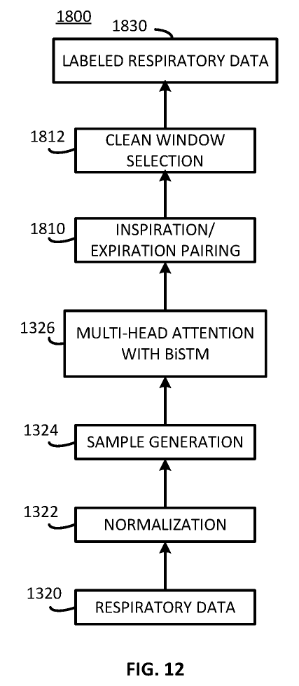

In one aspect, the unique Inspiration/Expiration Pairing (e.g. 1810 in FIG. 12) and Window Selection process (e.g. 1812 in FIG. 12) may help ensure that the predictions are as accurate as possible.

Among other aspects, at least some examples of the present disclosure may perform labeling of expiratory pause (as previously described) whereas at least some prior techniques may be limited solely to identifying and labeling inhalations, which may significantly limit the extent to which timing decisions may be leveraged from the respiratory signal. This novel labeling of the expiratory pause (according to at least some examples of the present disclosure) may enable calculating, among other things, the end of expiration for an overall more accurate respiratory phase determination.

As shown in diagram 1800 of FIG. 12, in some examples the inference workflow (e.g. using a constructed data model as in FIG. 5 to determine respiratory phase information) may comprise at least some postprocessing aspects, which may comprise inspiration/expiration pairing (1810 in FIG. 12) through the selection of the highest confidence value per group of model output above a threshold, and then selecting the highest Inspiration confidence value (if multiple exist) between each Expiration confidence value. Thereafter, the process is applied in a reverse manner such as selecting the highest Expiration confidence value between each Inspiration value. Next, the method selects the highest Expiration Pause confidence value between each Expiration to Inspiration. Finally, the paired inspiration to expiration timings may be forced to be within a specific duration length, and any such timings outside of the duration (e.g. the timing would not correspond to a nominal duration of a breath or portion thereof) would be removed. Accordingly, the Inspiration/Expiration pairing post-processing and clean window selection (1812 in FIG. 12) ensures that only the most accurate prediction labels (e.g. predictions of changepoints (e.g. transitions) between different respiratory phases) remain after such processing.

In some examples, the clean window selection 1812 in FIG. 12 may comprise calculating a frequency band of 1 to 10 Hz for a window, transforming a root mean square (RMS), and/or implementing a logistic regression classifier.

With further reference to FIG. 12, this inference mapping via the actions (e.g. 1322, 1324, 1326, 1810, 1812 in FIG. 12) relative to respiratory data (e.g. respiratory signa 1020 in FIG. 7; 1320 in FIG. 10) may culminate in labeled respiratory data 1830, as shown in FIG. 12, where the labeled respiratory data 1830 may correspond to the diagram 2500 in FIG. 14, as further described below.

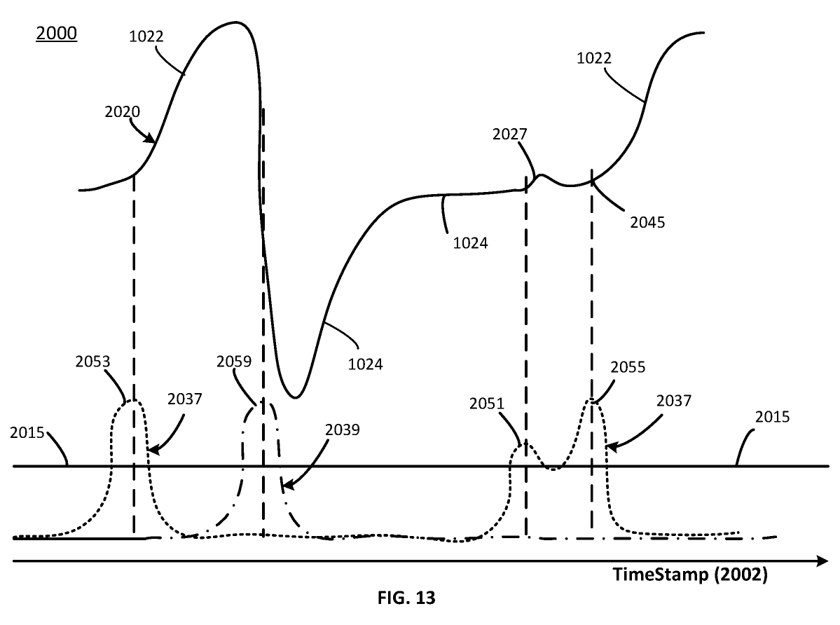

This process is further illustrated in the diagram 2000 of FIG. 13, which depicts a respiratory signal 2020, as well as dotted line 2037 depicting a model output confidence for inspiration and dashed-dotted line 2039 depicting a model output confidence for expiration. In some examples, a first step may comprise, for each continuous grouping of confidences above a threshold 2015, selecting a timestamp 2002 having the maximum confidence. As part of this process, in some instances, a “false positive” inspiration may be detected such as at 2051, due to a temporary upward movement (at 2027) in the respiration signal. In some examples, a second step may comprise, between each expiration (e.g. 2059), selecting those inspiration timestamps having the maximum confidences (e.g. at 2053, 2055), leading to the removal of the false positive (at 2051 (e.g. 2027 in the respiratory signal 2020) and the identification of the inspiration timestamp 2055 which corresponds to the genuine onset of the inspiratory phase (e.g. at 2045 in respiratory signal 2020).

FIG. 14A includes a diagram 2500 which combines the information developed per the diagrams of FIGS. 7-13. In one aspect, diagram 2500 may illustrate how, via the construction of the data models (e.g. FIGS. 3-4) and use of the constructed data models (e.g. FIG. 5), a relatively high correspondence between human labeled transitions and prediction labeled transitions. The human labeled transitions are shown at dashed-dotted lines 1112 (e.g. start of inspiration (i.e. expiratory pause to inspiration transition), at dashed-dotted lines 1122 for expiration (i.e. inspiration to expiration transition), and at dashed-dotted lines 1132 for expiratory pause (i.e. expiration to expiratory pause transition)). Meanwhile, the prediction labeled transitions are shown at dotted lines 2512 for start of inspiration (i.e. expiratory pause to inspiration transition), at dotted lines 2522 for expiration (i.e. inspiration to expiration transition), and at dotted lines 2532 for expiratory pause (i.e. expiration to expiratory pause transition). While there is a small difference between the human label (1132) for expiratory pause and the predicted label (2532) for expiratory pause, the predicted label (2532) is close enough to the human label (1132) that the predicted label (2532) can be adopted and used to designate the start of the expiratory pause 1026 and/or end of expiration 1024.

With this in mind, it can be seen that, via at least some example methods of the present disclosure which employ use of constructed data models, automated labeling of transitions between different respiratory phases can be made accurately and robustly such that the laborious, time-consuming human labeling of such transitions can be omitted in some instances. Moreover, in addition to making the basic determination of respiratory phase information, this determined information may be further exploited to facilitate patient care in various ways.

With this in mind, FIG. 14B is a block diagram representing example implementation portion 2800 comprising methods which use the outputs determined from applying an example constructed data model.

As shown at 2820 in FIG. 14B, in some examples the determination of respiratory phase information per at least some examples of the present disclosure may be used in example methods of determining upper airway collapse. For instance, the example method may isolate inhalations (e.g. inspiratory phase) during sleep and optionally isolate such inhalation (e.g. inspiratory phase) during respiration events such as (but not limited to) sleep disordered breathing (SDB) events, which may comprise obstructive sleep apnea (OSA) events and/or other events. In some such examples, the inhalations identified during respiration events and/or during sleep may be used as an input to an automated collapse pattern detection method. For instance, the example automated collapse pattern detection method may be used to identify a complete concentric collapse ( CCC) pattern among other upper airway (UA) collapse patterns. In one aspect, this identification also may comprise outputting a confidence in the identification of the CCC pattern, a True/False designation of the potential identification, and/or a confidence/classification of the type/location of airway collapse.

As shown at 2825 in FIG. 14B, in some examples the determination of respiratory phase information per at least some examples of the present disclosure may be used in example methods of automatically determining, adjusting, and/or tracking the sensing settings of a medical device (e.g. implantable and/or external), which may comprise respiration sensing settings in some examples. For instance, a medical device may comprise a sensing element (e.g. accelerometer, impedance, other) for which sensing data is known for respiration information (e.g. timing of inspiration and/or other phases) and which comprise device sensing settings for obtaining this respiration information. In some examples, the sensing settings of this device may be calibrated to be more accurate by using the determinable outputs from a constructed data model (e.g. FIGS. 3-5), wherein the constructed data model is based on the known input(s) from a highly accurate data source such as (but not limited to) a first respiratory sensing session (e.g. PSG data or other sleep study) (e.g. 400 in FIG. 2). As previously noted, this highly accurate data source may comprise externally measured breathing information (e.g. airflow). Via the calibration of the sensing element of the medical device, an ideal set of sensing parameters for the sensing element (e.g. accelerometer) of the medical device may be determined and applied to detect breath timings.

In some examples, the above-described adjustment of the sensing settings for the medical device may be performed in a complementary manner with therapy settings 2830 (FIG. 14B) of the medical device such as (but not limited to) therapy settings (e.g. amplitude, duration, timing, etc.) for treating respiratory ailments such as (but not limited to) sleep disordered breathing (SDB) (e.g. obstructive sleep apnea). The therapy settings 2830 may comprise electrical stimulation therapy settings for upper airway patency-related tissue (e.g. hypoglossal nerve, infrahyoid muscle (IHM)-innervating nerve and/or muscles, etc.), therapy settings for mechanical ventilation, therapy settings for positive airway pressure (PAP) devices, etc. Such adjustments may improve patient outcomes (e.g. reduce disease burden (e.g. AHI)) via enhancing therapy efficacy.

As shown at 2835 in FIG. 14B, in some examples the determination of respiratory phase information per at least some examples of the present disclosure may be used in example methods of determining additional respiratory metrics. At least some additional respiratory metrics may comprise “Breaths lost” during apnea/hypopnea, Tidal volume, Expiratory Volume, etc.

At shown at 2840 in FIG. 14B, in some examples the determination of respiratory phase information per at least some examples of the present disclosure may be used in example methods of validating sensing protocols such as determining the efficacy of sensing respiration (e.g. phase) transitions, timings, etc. when just suboptimal data is available.

At shown at 2845 in FIG. 14B, in some examples the determination of respiratory phase information (and/or other respiratory information, patterns, irregularities, etc.) per at least some examples of the present disclosure may be used in example methods of diagnosing respiratory diseases, which may occur earlier than might otherwise be observed in existing clinical pathways.

At shown at 2850 in FIG. 14B, in some examples the determination of respiratory phase information (and/or other respiratory information) per at least some examples of the present disclosure may be used in example methods of optimizing breathing techniques for a variety of athletic and/or fitness applications. One such example application may comprise breathing training for underwater diving and/or breathing-intensive activities, situations, etc.

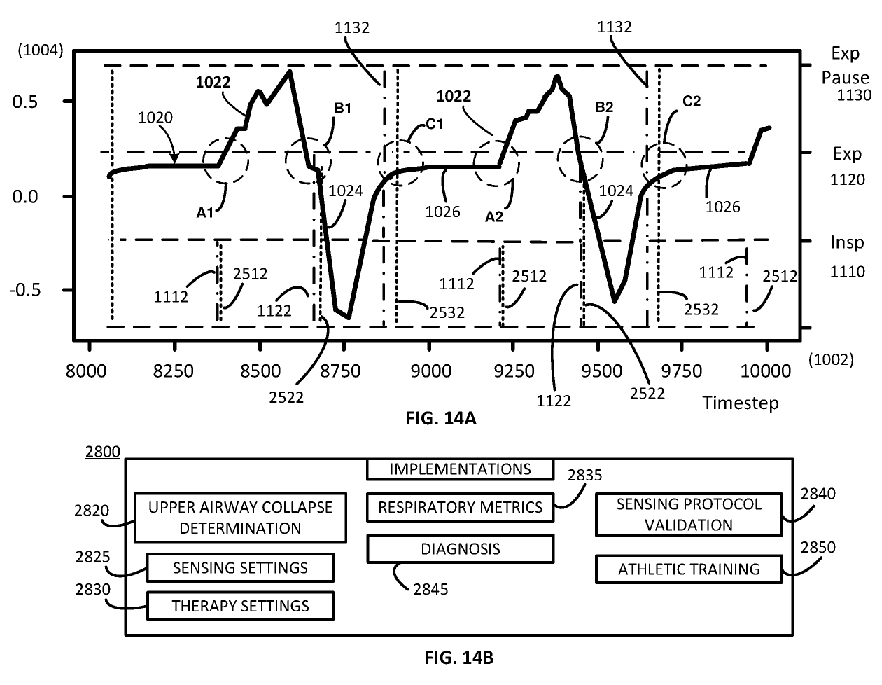

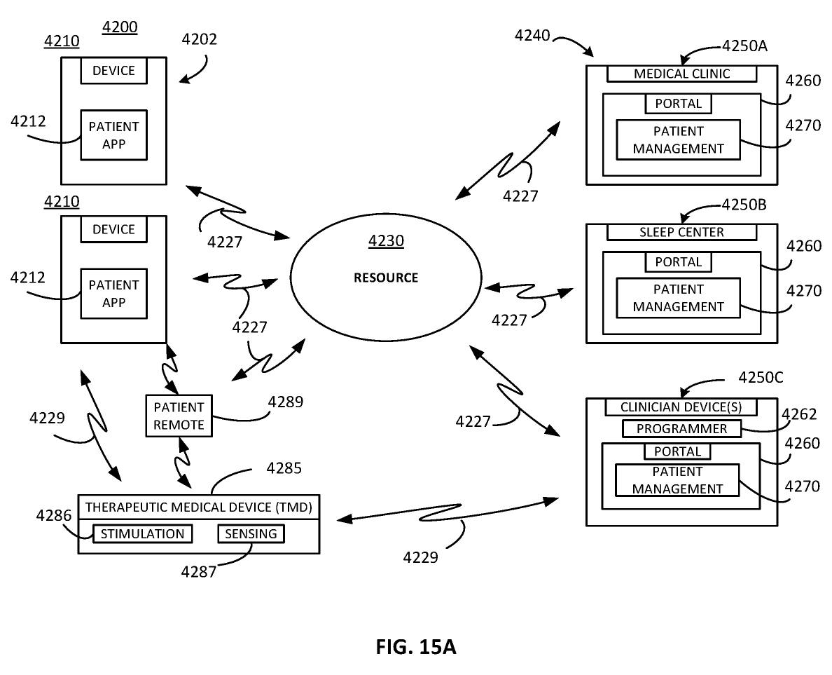

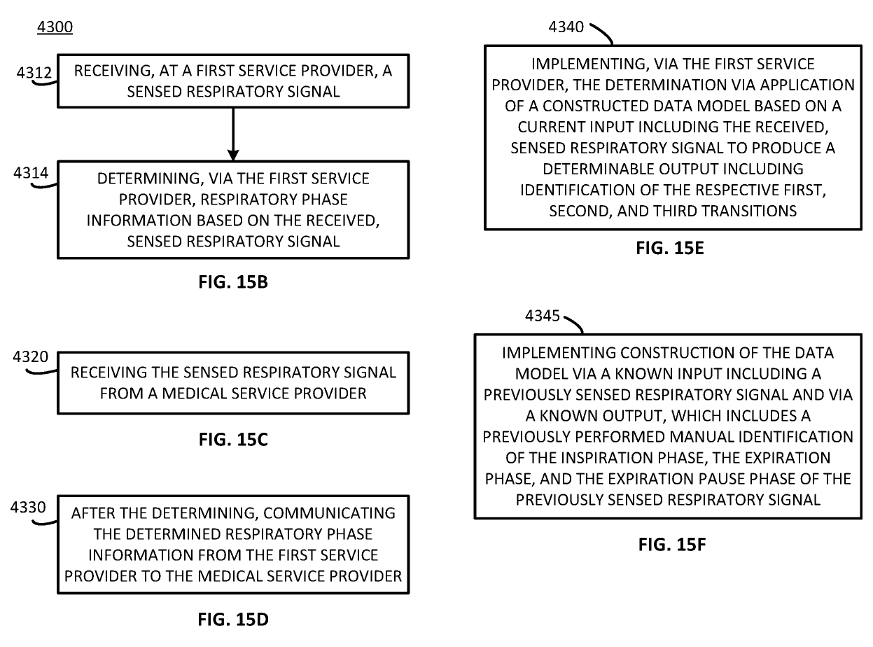

FIG. 15A is a block diagram schematically representing an example arrangement 4200, deployable in an example method of (or as an example system for) to facilitate SDB patient care such as within (but not limited to) a patient management arrangement.

In some examples, the example arrangement 4200 may comprise a resource 4230, which comprises a computing resource (including stored programming) provided via a third party (third party service provider) to help provide and support the at least some aspects of the example methods throughout the present disclosure such as (but not limited to) a SDB patient care environment such as (but not limited to) at least some aspects of the example methods and/or devices of FIGS. 1-14B, 15B-17 throughout the present disclosure. In some such examples, the resource 4230 may sometimes be referred to as a service provider resource. In some examples, resource 4230 may comprise at least a portion or, and/or an example implementation of, the control portion 4500 (FIG. 16A) and user interface 4540 (FIG. 16C). In some examples, the third party providing resource 4230 may comprise a device manufacturer, device supplier, or third party contracted by a device manufacturer. The resource 4230 may be hosted via the internet, World Wide Web, and/or other network communication link. Via a wireless communication protocol as represented by directional arrows 4227 and/or wired communication protocol, the resource 4230 may communicate with, and/or support communication among various other parties 4240 such as (but not limited to) a medical clinic 4250A, a sleep center 4250B, clinician device(s) 4250C (e.g. programmer 4262 and/or portal 4260), therapeutic medical device (TMD) 4285, patient remote 4289, and/or patient devices 4210.

With further reference to FIG. 15A, each of the entities associated with the respective devices 4250A, 4250B, 4250C may work together in at least some aspects to help coordinate care for the patient(s). Each entity may provide a particular form of expertise in patient care, such examples in which one entity may comprise a medical clinic (4250A), while another entity (associated with a different device 4250B) may comprise a sleep center, and some other entities comprise a clinician (4250C). Yet other entities may comprise providers which support patient care in some manner. There may be greater or fewer than the various devices (e.g. care entities) 4250A, 4250B, 4250C shown in FIG. 14B which form at least part of a care team.

Each of the devices 4250A, 4250C, 4250C comprise a computing resource, such as workstation or other computing device which may be stationary or mobile. In some examples, at least one of the respective devices comprises a user interface (GUI) to facilitate display a workflow, inputs, outputs, etc. relative to perform a method which implements a SDB patient care environment such as (but not limited to) the examples of FIGS. 1-14B, 15B-17. The user interface may comprise one example implementation of, and/or comprise at least some of the features and attributes of, the user interface 4540 described later in association with at least FIGS. 16C. Accordingly, it will be further understood that the various devices 4250A, 4250B, 4250C may comprise a control portion, or comprise an example implementation of one part of a control portion, such as control portion 4500 (FIGS. 16A-17).

As part of this arrangement, the devices may display at least some patient information (described throughout various examples of the present disclosure) as part of preparing and/or executing such methods, as well as caring for a patient within a SDB patient environment via various examples of the present disclosure. In some examples, these devices may participate in training (e.g. constructing) a data model, updating a data model, selecting and/or entering inputs to a data model, selecting and/or reporting outputs of a data model, modifying inputs/outputs of a data model, etc. At least some of these actions may comprise receiving patient information (according to various examples of the present disclosure), inputting patient information, and/or sending patient information to the resource 4230 and/or to other devices in arrangement 4200 via resource 4230 or directly.

With continued reference to FIG. 15A, in some examples, among other functions and features, the portal 4260 of devices 4250A, 4250B, 4250C, etc. may comprise a patient management app 4270 by which the particular care provider (e.g. medical clinic, sleep center, clinician, etc.) may manage patient care among a group of patients, for an individual patient, etc. In some such examples, this patient care may comprise implementing at least some aspects of the various example methods throughout the present disclosure. In some examples, when deployed in relation to the respective devices 4250A, 4250B, 4250C, the portal 4260 may sometimes be referred to as a medical clinic portal, sleep center portal, clinician portal, respectively.

The patient management app 4270 also may enable communicating with other entities (e.g. 4250A, 4250B, 4250C) regarding patient care of the patients associated with devices 4210. In some examples, the clinician devices 4250A, 4250B, etc. may communicate with each other via at least resource 4230 (e.g. network communication link, internet, web, etc.) as represented via indicators 4227.

With further reference to FIG. 15A, in some examples a clinician device 4250C comprises a programmer 4262 for programming, etc. the TMD 4285. For example, the programmer 4262 may periodically communicate with the TMD 4285 (or external medical device) wirelessly (e.g. inductive telemetry) to initially configure and/or later modify the configured stimulation therapy settings, sensing settings, therapy management settings, etc. of the TMD 4285, as well as send/receive patient information to and from the TMD 4285. However, in some examples, the programmer 4262 which may perform tasks or operations (relating to SDB patient care, maintenance) etc. in addition to programming stimulation-related aspects and/or sensing-related aspects of the TMD 4285.

As shown in FIG. 15A, example arrangement 4200 also may comprise an array 4202 of computing devices 4210, each of which host a patient app 4212 relating to patient care. The devices 4210 may sometimes be referred to as patient devices, patient computing devices, and the like. The patient app 4212 may provide patient education and/or enable communication with resource 4230, and/or communicate (via resource 4230 and/or directly) with any one of the devices 4250A, 4250B, 4250C. Via such communication, the patient app 4212 may receive, send, display, store, and/or communicate patient information, usage information, etc. In some examples, the patient app 4212 is one vehicle via which a patient may participate in a SDB patient care path according to the various examples of the present disclosure. In some such examples, at least some of the patient devices 4210 may comprise a mobile computing device, such as a mobile phone, tablet, smartwatch, etc. which has a user interface 4540 (FIG. 18C) to provide for operation of, and display of, the patient app 4212.

In some examples, the devices 4250A, 4250B, 4250C (with or without patient app 4212) and/or devices may communicate results of a home sleep study which provides a sensed respiratory signal to service provider in order to identify respiratory phase information (e.g. transitions between respiratory phases) according to various examples throughout the present disclosure for use in various purposes related to SDB patient care.

As shown in FIG. 15A, in some examples the example arrangement 4200 may comprise a therapeutic medical device (TMD) 4285, which include implantable components and/or external components. In some examples, the TMD 4285 may be adapted for treating sleep disordered breathing (SDB) and/or other patient conditions (e.g. cardiac, etc.). A patient remote control 4289 may communicate with the TMD 4285 via a wireless communication protocol either directly or indirectly via an intermediary communication element (e.g. antenna, other). In some examples, such wireless communication may take the form of inductive telemetry. In some examples, the TMD 4285 may comprise an implantable pulse generator (IPG) for generating stimulation therapy signals to be delivered to the patient via a stimulation element (e.g. electrode) within the patient.

With further reference to FIG. 15A, in general terms, the patient remote control 4289 enables a patient to have limited control over their stimulation therapy, such as turning the stimulation therapy on/off, pause, and/or increasing or decreasing the amplitude of stimulation within a lower and upper limit set by a clinician (and/or device manufacturer, supplier, etc.). In some examples, the patient remote control 4289 also tracks patient usage of these controls to enable a clinician, the patient, and others to learn about the patient’s usage, therapy effectiveness, patient adherence, etc. In some examples, the patient remote control also may receive some information from the TMD 4285 regarding stimulation metrics, sensing metrics, therapy metrics, usage metrics, etc.

In some examples, the patient remote control 4289 is in communication with the patient app 4212 such that patient app 4212 on device 4210 may receive the patient usage information from the patient remote control 4289, as well as whatever therapy, sensing, etc. information was communicated from the TMD 4285 to the patient remote control 4289. In some examples, the communication between the patient remote control 4289 and the patient app 4212 (on patient device 4210) may occur wirelessly 4229 via a number of wireless communication protocols such as, but not limited to, a Bluetooth® wireless communication protocol. In some examples, the communication between patient remote control 4289 and patient app 4212 (on patient device 4210) may occur via a wired connection.

As noted here and elsewhere, the patient app 4212 may communicate this information (received from the patient remote control 4289) to one or more of the devices 4250A, 4250B, 4250C via resource 4230 to facilitate patient management according to examples of the present disclosure. In some examples, the patient app 4212 also may obtain some patient information through the patient’s use of the app 4212 which also may be communicated to the devices 4250A, 4250B, 4250C separately from, or integrated with, the patient usage information and therapy information from the patient remote control 4289 and/or TMD 4285. In some such examples, this communicated information may comprise sleep study information (e.g. formal (e.g. PSG), home, etc.) for use in the manner described throughout various examples of the present disclosure.

As further shown in FIG. 15A, in some examples a TMD 4285 may comprise a stimulation component 4286 and/or sensing component 4287. In some examples, the stimulation component 4286 comprises a stimulation engine to generate a stimulation signal to be applied to a tissue (e.g., nerve, muscle, etc.). In the examples in which the TMD 4285 comprises an implantable pulse generator (IPG), the tissue to be stimulated may comprise tissue to maintain or restore upper airway patency, such as but not limited to a hypoglossal nerve, infrahyoid-muscle (IHM)-innervating nerve, and/or other nerves/muscles which may directly or indirectly promote upper airway patency. In some such examples, the stimulation component 4286 also may comprise circuitry for generating and delivering the stimulation signal. In some examples, the stimulation component 4286 of the TMD 4285 also may comprise a stimulation element, such as an electrode through which the stimulation signal may be applied to the target tissue.

In some examples, the sensing component 4287 comprises a sensing engine to receive a sensing signal obtained relative to a tissue (e.g., muscle, organ, etc.). In the examples in which the TMD 4285 comprises an IPG for treating sleep disordered breathing (SDB), the tissue to be sensed may be related to respiration, oxygenation, cardiac functions, upper airway patency, and the like. In some such examples, the sensing component 4287 also may comprise circuitry for receiving and processing the sensing signal. In some examples, the sensing component 4287 of the TMD 4285 also may comprise a sensing element, such as an electrode or other element through which the sensing signal is obtained. In some examples, the sensing element may comprise an accelerometer for determining sleep information, respiratory information, posture information, physical control information. The accelerometer may be implantable, and in some examples, may be incorporated within a device including a stimulation generating element, such as an implantable pulse generator.

In some examples, the stimulation component 4286 and/or sensing component 4287 may be on-board the TMD 4285, which in some examples may comprise a microstimulator which houses stimulation circuitry, power element, and/or communication element.

In some examples, at least a portion of the stimulation component 4286 and/or sensing component 4287 may be separate from, and independent of, a housing of the TMD 4285 with one or both components 4286, 4287 being in wired or wireless communication with the TMD 4285.

FIGS. 15B-15F are diagrams schematically representing methods and/or aspects of methods of determining respiratory phase information based on a respiratory signal. In some examples, the various methods of FIGS. 15B-15F may be implemented via a device configured (e.g. programmed) to perform the various actions. In some examples, the methods of FIGS. 15B-15F may comprise at least some of substantially the same features as, and/or an example implementation of, the various examples of the present disclosure as described in association with at least FIGS. 1-14B, 16A-17.

As shown at 4312 in FIG. 15B, in some examples, method 4300 comprises receiving, at a first service provider, a sensed respiratory signal of a first respiratory sensing session, and at 4314, method 4300 comprises determining, at the first service provider, respiratory phase information based on the received sensed respiratory signal.

As shown at 4320 in FIG. 15C, in some examples method 4300 also may comprise receiving the sensed respiratory signal from a medical service provider. In some such examples, the respiratory signal may be sensed via an airflow sensor.

As shown at 4330 in FIG. 15D, in some examples method 4300 also may comprise after the determining (e.g. at 4314 in FIG. 15B), communicating the determined respiratory phase information from the first service provider to the medical service provider.

As shown at 4340 in FIG. 15E, in some examples method 4300 also may comprise implementing, via the first service provider, the determination via application of a constructed data model based on a current input including the received, sensed respiratory signal to produce a determinable output including identification of the respective first, second, and third transitions.

As shown at 4345 in FIG. 15F, in some examples method 4300 also may comprise implementing construction of the data model via a known input including a previously sensed respiratory signal and via a known output, which includes a previously performed manual identification of the inspiration phase(s), the expiration phase(s), and/or the expiration pause phase(s) of the previously sensed respiratory signal.

FIG. 16A is a block diagram schematically representing an example control portion 4500. In some examples, control portion 4500 provides one example implementation of a control portion forming a part of, implementing, and/or generally managing example methods, data models (e.g. training, operation), patient information, medical devices (implantable and/or external), sensing (e.g. portions, element(s)), stimulation (e.g. portion, elements), power/control elements (e.g. pulse generators), communication elements/pathways, devices, user interfaces, instructions, information, engines, elements, functions, and/or actions, etc., as described throughout examples of the present disclosure.

In some examples, control portion 4500 includes a controller 4502 and a memory 4510. In general terms, controller 4502 of control portion 4500 comprises at least one processor 4504 and associated memories. The controller 4502 is electrically couplable to, and in communication with, memory 4510 to generate control signals to direct operation of at least some of the example methods, data models (e.g. training, operation), patient information, medical devices (implantable and/or external), sensing (e.g. portions, element(s)), stimulation (e.g. portion, elements), power/control elements (e.g., pulse generator), communication elements/pathways, devices, user interfaces, instructions, information, engines, elements, functions, and/or actions, etc., as described throughout examples of the present disclosure. In some examples, these generated control signals include, but are not limited to, employing instructions 4511 and/or information 4512 stored in memory 4510 to implement the example methods and/or devices according to the various examples of the present disclosure which generally relate to SDB patient care. In some instances, the controller 4502 or control portion 4500 may sometimes be referred to as being programmed to perform the above-identified actions, functions, etc. such that the controller 4502, control portion 4500 and any associated processors may sometimes be referred to as being a special purpose computer, control portion, controller, or processor. In some examples, at least some of the stored instructions 4511 and/or information 4512 may form at least part of, and/or, may be referred to as an engine.

In response to or based upon commands received via a user interface (e.g., user interface 4540 in FIG. 16C) and/or via machine readable instructions, controller 4502 generates control signals as described above in accordance with at least some of the examples of the present disclosure. In some examples, controller 4502 is embodied in a general purpose computing device while in some examples, controller 4502 is incorporated into or associated with at least some of the example methods, data models (e.g. training, operation), patient information, medical devices (implantable and/or external), sensing (e.g. portions, element(s)), stimulation (e.g. portion, elements), power/control elements (e.g., pulse generator), communication elements/pathways, devices, user interfaces, instructions, information, engines, elements, functions, and/or actions, etc., as described throughout examples of the present disclosure.

For purposes of this application, in reference to the controller 9802, the term “processor” shall mean a presently developed or future developed processor (or processing resources) that executes machine readable instructions contained in a memory. In some examples, execution of the machine readable instructions, such as those provided via memory 4510 of control portion 4500 cause the processor to perform the various IMD operations and/or related operations, as generally described in (or consistent with) at least some of the various examples of the present disclosure. The machine readable instructions may be loaded in a random access memory (RAM) for execution by the processor from their stored location in a read only memory (ROM), a mass storage device, or some other persistent storage (e.g., non-transitory tangible medium or non-volatile tangible medium), as represented by memory 4510. In some examples, the machine readable instructions may comprise a sequence of instructions, a processor-executable machine learning model, or the like. In some examples, memory 4510 comprises a computer readable tangible medium providing non-volatile storage of the machine readable instructions executable by a process of controller 4502. In some examples, the computer readable tangible medium may sometimes be referred to as, and/or comprise at least a portion of, a computer program product. In other examples, hard wired circuitry may be used in place of or in combination with machine readable instructions to implement the functions described. For example, controller 4502 may be embodied as part of at least one application-specific integrated circuit (ASIC), at least one field-programmable gate array (FPGA), and/or the like. In at least some examples, the controller 4502 is not limited to any specific combination of hardware circuitry and machine readable instructions, nor limited to any particular source for the machine readable instructions executed by the controller 4502.

In some examples, processing resources and/or other components of the control portion 4500 may comprise and/or may be implemented via graphics processing resources such as (but not limited to) a graphics processing unit (GPU), accessible via the web, cloud, and/or other pathways. In some such examples, example methods of the present disclosure may be highly parallelizable and scalable, while providing high levels of accuracy.

In some examples, control portion 4500 may be entirely implemented within or by a stand-alone device.

In some examples, the control portion 4500 may be partially implemented in or via one of the example methods, data models (e.g. training, operation), patient information, medical devices (implantable and/or external), sensing (e.g. portions, element(s)), stimulation (e.g. portion, elements), power/control elements (e.g., pulse generator), communication elements/pathways, devices, user interfaces, instructions, information, engines, elements, functions, and/or actions, etc. and partially implemented in a computing resource (e.g., at least one external resource) separate from, and independent of, the various example devices (or portions thereof) but in communication with the treatment devices (or portions thereof). For instance, in some examples control portion 4500 may be implemented via a server accessible via the cloud and/or other network pathways. In some examples, the control portion 4500 may be distributed or apportioned among multiple devices or resources such as among a server, a treatment device (or portion thereof), and/or a user interface.

In some examples, control portion 4500 includes, and/or is in communication with, a user interface 4540 as shown in FIG. 16C.

FIG. 16B is a diagram schematically illustrating at least some example arrangements of a control portion 4520 by which the control portion 4500 (FIG. 16A) can be implemented, according to one example of the present disclosure. In some examples, control portion 4520 is entirely implemented within or by an MD 4525, which has at least some of substantially the same features and attributes as a medical device as previously described throughout the present disclosure. In some examples, control portion 4520 is entirely implemented within or by a remote control 4519 (e.g., a programmer) external to the patient’s body, such as a patient control 4532 and/or a physician control 4534. In some examples, the control portion 4500 is partially implemented in the MD 4525 and partially implemented in the remote control 4519 (at least one of patient control 4532 and physician control 4534). In some examples, the control portion 4520 in FIG. 16B is at least partially implemented via a clinician portal (e.g., 4662 in FIG. 17), which may or may not be in complementary relation with elements 4525 and 4519.

FIG. 16C is a block diagram schematically representing user interface 4540, according to one example of the present disclosure. In some examples, user interface 4540 forms part of (and/or is accessible via) a device external to the patient and by which the therapy system may be at least partially controlled and/or monitored. The external device which hosts user interface 4540 may be a patient remote (e.g., 4532 in FIG. 16B), a physician remote (e.g., 4534 in FIG. 16B) and/or a clinician portal (e.g., 4662 in FIG. 17). In some examples, user interface 4540 comprises a user interface or other display that provides for the simultaneous display, activation, and/or operation of at least some of the example methods, data models (e.g. training, operation), patient information, medical devices (MDs), sensing (e.g. portions, element(s)), stimulation (e.g. portion, elements), power/control elements (e.g., pulse generator), communication elements/pathways, devices, user interfaces, instructions, information, engines, elements, functions, and/or actions, etc. In some examples, at least some portions or aspects of the user interface 4540 are provided via a graphical user interface (GUI) and may comprise a display 4544 and input 4542.

FIG. 17 is a diagram 4600 which schematically represents some example implementations by which a medical device 4610 may communicate wirelessly with devices outside the patient. In some examples, the medical device 4610 may comprise at least some implantable components and/or at least some external components.

As shown in FIG. 17, in some examples, the medical device 4610 may communicate with at least one of a patient app 4619 on a mobile device 4620, a patient remote control 4640, a clinician programmer 4650, and a patient management tool 4660. The patient management tool 4660 may be implemented via a cloud-based portal 4662, the patient app 4619, and/or the patient remote control 4640, each of which may comprise a user interface (e.g., having a display, input) such as user interface 4540 in FIG. 16C.

Among other types of data, these communication arrangements enable the medical device 4610 to communicate, display, adjust, manage, etc. any of the example methods, data models (e.g. training, operation, etc.) commands, etc. in association with any of the various examples of the present disclosure.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein.

1. A method comprising:
receiving, at a first service provider, a sensed respiratory signal of a first respiratory sensing session; and
determining, at the first service provider, respiratory phase information based on the received sensed respiratory signal.

2. The method of claim 1, wherein the receiving comprises receiving the sensed respiratory signal from a medical service provider, and after the determining, communicating the determined respiratory phase information from the first service provider to the medical service provider.

3. The method of claim 1, wherein the first respiratory sensing session comprises a formal sleep study including polysomnographic information.

4. The method claim 1, wherein the determining comprises:
detecting each change point of a plurality of change points in the sensed respiratory signal, wherein each change point includes:
a change in a direction of the sensed respiratory signal; and
a magnitude of the change exceeding a selectable first threshold.

5. The method of claim 4, wherein the determining comprises:
classifying each change point as at least one of:
a first transition from an inspiration phase to an expiration phase;
a second transition from the expiration phase to an expiration pause phase; and
a third transition from the expiration pause phase to the inspiration phase.

6. The method of claim 5, comprising:
implementing, via the first service provider, the determination via application of a constructed data model based on a current input of the received sensed respiratory signal to produce a determinable output including identification of the respective first, second, and third transitions in the received, sensed respiratory signal.

7. The method of claim 6, comprising wherein the received, sensed respiratory signal is obtained from an airflow sensor.

8. The method of claim 6, further comprising:
implementing construction of the constructed data model via:
a known input, which includes a previously sensed respiratory signal; and
a known output, which includes a previously performed manual identification of the inspiration phase, the expiration phase, and the expiration pause phase of the previously sensed respiratory signal.

9. The method of claim 8, wherein the manual identification includes identification of at least one of
the first transition from the inspiration phase to the expiration phase;
a second transition from the expiration phase to the expiration pause phase; and
a third transition from the expiratory pause phase to the inspiration phase.

10. The method of claim 8, wherein the previously performed manual identification comprises the detecting and the classifying of each change point of the previously sensed respiratory signal.

11. A first service provider for providing sleep disordered breathing care, the first service provider comprising:
a control portion programmed to:
receive, from a medical service provider, a sensed respiratory signal of a first respiratory sensing session; and
determine, at the first service provider, respiratory phase information based on the received sensed respiratory signal.

12. The device of claim 11, wherein the control portion is programmed to receive the sensed respiratory signal from a medical service provider, and after the determining, communicate the determined respiratory phase information from the first service provider to the medical service provider.

13. The device of claim 11, wherein the first respiratory sensing session comprises a formal sleep study including polysomnographic information.

14. The device method claim 11, wherein the control portion is programmed to implement the determining to comprise:
detecting each change point of a plurality of change points in the sensed respiratory signal, wherein each change point includes:
a change in a direction of the sensed respiratory signal; and
a magnitude of the change exceeding a selectable first threshold.

15. The device of claim 14, wherein the control portion is programmed to implement the determining to comprise:
classifying each change point as at least one of:
a first transition from an inspiration phase to an expiration phase;
a second transition from the expiration phase to an expiration pause phase; and
a third transition from the expiration pause phase to the inspiration phase.

16. The device of claim 15, wherein the control portion is programmed to implement, via the first service provider, the determination via application of a constructed data model based on a current input of the received sensed respiratory signal to produce a determinable output including identification of the respective first, second, and third transitions in the received, sensed respiratory signal.

17. The device of claim 16, wherein the received, sensed respiratory signal is obtained from an airflow sensor.

18. The device of claim 16, wherein the control portion is programmed to implement construction of the constructed data model via:
a known input, which includes a previously sensed respiratory signal; and
a known output, which includes a previously performed manual identification of the inspiration phase, the expiration phase, and the expiration pause phase of the previously sensed respiratory signal.

19. The device of claim 18, the control portion is programmed to perform the manual identification to include identification of at least one of
the first transition from the inspiration phase to the expiration phase;
a second transition from the expiration phase to the expiration pause phase; and
a third transition from the expiratory pause phase to the inspiration phase.

20. The device of claim 18, wherein the previously performed manual identification comprises the detecting and the classifying of each change point of the previously sensed respiratory signal.
